(19) United States
(12) Patent Application Publication
(10) Pub. No.: US 2025/0025999 A1
(43) Pub. Date: Jan. 23, 2025

(54) Soft Robotic Modular and Reconfigurable Actuator

(71) Applicant: National University of Singapore, Singapore (SG)

(72) Inventors: AJINKYA SARANG BHAT, SINGAPORE (SG); CHEN HUA YEOW, SIGAPORE (SG)

(21) Appl. No.: 18/709,661

(22) PCT Filed: Nov. 11, 2022
(86) PCT No.: PCT/SG2022/050820

§ 371 Date: May 13, 2024

(30) Foreign Application Priority Data
Nov. 11, 2021 (SG) 10202112568Y

(51) Int. Cl. B25J 9/00; A61F 2/50; A61F 2/74; A61H 3/00; B25J 9/14; F16J 3/04

(52) U.S. Cl. CPC B25J 9/0006; A61F 2/74; A61H 3/00; B25J 9/142; F16J 3/04; A61F 2002/501; A61H 2201/1238

(57) ABSTRACT

The present invention describes soft robotic actuators (300,400). Each robotic actuator (300,400) is made up of a corrugated sleeve (110,110a,210,210a), an expandable blabber (120,220) and a lock member (370,480). The corrugated sleeve (110,110a,210,210a) has a hollow passageway (111,211) with a plurality of folds (112,212) extending along the passageway, with the hollow passageway extending along a length of the corrugated sleeve. The expandable blabber (120,220) is detachably inserted inside the hollow passageway (111,211) and being kept inside the corrugated sleeve (110,110a,210,210a) by the lock member (370,480). In a use application, a fluid medium is communicable into the bladder under pressure through a tubing opening formed on the lock member (370,480) results in reversible inflation of the bladder (120,220). Inflation of the bladder causes the bladder to press against and deform the corrugated sleeve, thereby generating a force output and controlled extension/bending of the robotic actuators (300,400).

### RELATED APPLICATIONS

The present invention claims priority to Singapore patent application Ser. No. 10202112568Y filed on 11 Nov. 2021, the disclosure of which is incorporated in its entirety.

### FIELD OF INVENTION

The present disclosure relates to soft robotic actuators; these robotic actuators are made of modular components and are thus reconfigurable. Each of the modular components may differ in mechanical and/or dimensional properties from one another; different units of these modular components can be freely combined into subassemblies and are reconfigured to form the soft robotic actuators, which controllably perform a desired motion of extension and/or bending, and to deliver a desired force output.

### BACKGROUND

Soft Robotics is a class of robotics that involves creating compliant designs by use of either compliant mechanisms or materials. Soft pneumatic actuators are a major class of soft actuators that use compliant materials; they are gaining popularity due to their utility in healthcare, wearable robotics, surgery, locomotion, soft manipulation for delicate objects, and so on. Conventional Soft Pneumatic Actuators are manufactured from three primary materials, namely silicone polymers, thermoplastic polyurethane (TPU) and fabrics; these known pneumatic actuators use TPU for hermetic sealing of the pneumatic chambers. In isolation, each of these materials poses challenges. FIG. 1 shows the conventional materials that have been popular for designing soft pneumatic actuators and their properties. Each material has its advantages in terms of mechanical properties and manufacturability, but it comes with trade-offs. Silicone has been the most used material for manufacturing soft pneumatic actuators. It possesses high deformation properties due to its high strain limit, but its low Young's Modulus tends to limit force output in isolation. Methods of using fibre-reinforcement and external constraints have tried to improve the force output or programmability, but this leads to use of higher input pressure. TPU-based actuators are usually manufactured by 3D printing, making them more repeatable than other techniques for soft robot manufacturing. However, the increased stiffness of TPU leads to very high-pressure requirements, although TPU actuators provide large force output. These actuators also have the highest energy losses due to their high stiffness. The third class of actuators are made from TPU-backed fabrics; the fabrics provide conformance, while the TPU acts as a hermetic seal. The fabric actuators are usually manufactured by heat sealing TPU-TPU contact sides on two sheets back-to-back via ironing or ultrasonic welding. These designs have lower energy losses than TPU actuators, but their design complexity is limited due to their manufacturing technique. Heat seals are also prone to rupture at the seam, and thus, these actuators usually cannot tolerate pressures above substantially 150 kPa. In view of these limitations, there presents a need to develop soft robotics actuator carrying features and/or properties capable of addressing at least some of the shortcoming inherently exhibited in the prior arts discussed above.

### SUMMARY

The following presents a simplified summary to provide a basic understanding of the present invention. This summary is not an extensive overview of the present invention, and is not intended to identify key features of the invention. Rather, it is to present some of the inventive concepts of this invention in a generalised form as a prelude to the detailed description that is to follow.

The present invention seeks to provide soft robotic actuators which perform a desired motion of extension and/or bending, and to deliver a desired force output.

In one embodiment, the present invention provides a robotic actuator comprising: a corrugated sleeve formed with corrugating folds extending from a first end to a second end and defining an interior passageway; and a bladder being detachably disposed inside the interior passageway of the corrugated sleeve; wherein, when a fluid medium is supplied into the bladder, the bladder is inflated, thereby compressing against the interior passageway, deforming the corrugated sleeve and providing a force output at the robotic actuator, and the bladder is reversible deflated when the fluid medium is released.

In one embodiment, the corrugated sleeve is cylindrically shaped; in another embodiment, the corrugated sleeve is substantially hemi-cylindrical shaped and comprises a flat surface, resulting in the interior passageway being substantially hemi-cylindrical. Preferably, the bladder is shaped to fit substantially snugly into the interior passageway.

Preferably, the corrugating folds are spaced apart at a regular pitch distance. Preferably, a wall of each corrugating fold at both an outside surface and the inner passageway is curved or arched shaped. In another embodiment, a wall of each of the corrugating fold at the inner passageway is substantially flat. It is possible that a width of each corrugating fold at the outside surface has a bigger dimension than a width of the corrugating fold at the inner passageway. It is possible to vary a tensile stiffness of a material along the flat surface to control bending of the corrugated sleeve; alternatively, a fabric strip can be attached along the flat surface to control the bending.

Preferably, radial fissures are formed radiating from the inner passageway and extending to both ends of the corrugated sleeve.

Preferably, the robotic actuator further comprises a lock member to removeably secure the bladder inside the corrugated sleeve. In one embodiment, the lock member comprises a female connector being connected to an end of the corrugated sleeve and a male connector being connected to an end of the bladder, so that the female and male connectors are engageable or dis-engageable by a rotatory motion. In another embodiment, the lock member comprises a female connector being connected to an end of the corrugated sleeve and a male connector being connected to an end of the bladder, so that the female and male connectors are engageable or dis-engageable by a sliding motion.

Preferably, the bladder is made of a material of lower stiffness that a material of the corrugated sleeve, so that the bladder is hermetically sealed and is inflatable by the fluid medium, whilst the corrugated sleeve controls extension or bending of the robotic actuator caused by inflating the bladder. In one embodiment, the bladder material is silicone rubber, whilst the material of the corrugated sleeve is thermoplastic urethane (TPU).

In another embodiment, the present invention provides a method for configuring extension or bending of a robotic actuator, the method comprises: configuring a corrugated sleeve by forming corrugating folds to extend from a first end to a second end, and defining an interior passageway; and removeably securing a bladder inside the corrugated sleeve, wherein the bladder is hermitically sealed to receive a fluid medium, wherein the bladder is formed from a material of lower stiffness compared to a material of the corrugated sleeve; wherein, when the fluid medium is supplied into the bladder, inflation of the bladder acts on the interior passageway, thereby causing a force output and extension of the robotic actuator.

Preferably, the method further comprises forming fissures along the corrugated sleeve to alter the stiffness of the corrugated sleeve, so that the force output is increased and the robotic actuator bends towards location of the fissures. Additionally or alternatively, the method further comprises forming the corrugated sleeve with a flat surface to obtain a semi-cylindrical passageway, so as to reduce the bending stiffness of the corrugated sleeve towards the flat surface and the robotic actuator responds by bending towards the flat surface.

Preferably, removeably securing the bladder inside the corrugated sleeve comprises connecting a lock member to an end of the corrugated sleeve and an end of the bladder.

In yet another embodiment, the present invention provides a method for configuring an assistive mechanism using the above robotic actuator; the method comprises: inserting a bladder inside a corrugated sleeve, wherein the bladder is hermitically sealed to receive a fluid medium and forming the bladder from a material of lower stiffness compared to a material of the corrugated sleeve; removeably securing one end of the bladder to an associated end of the corrugated sleeve with a lock member; mounting an opposite end of the corrugated sleeve with another lock member; and securing each of the lock members to a limb constituting a joint; wherein inflating the bladder creates a force output, and resulting extension and bending of the robotic actuator creates an assistive motion about the joint, whilst deflating the bladder reverses motions of the robotic actuator.

Preferably, the joint is constituted with a prothesis or an exoskeleton.

### BRIEF DESCRIPTION OF THE DRAWINGS

This invention will be described by way of non-limiting embodiments of the present invention, with reference to the accompanying drawings, in which:

FIG. 1 illustrates characteristics of various materials usable for manufacturing a soft robotic actuator;

FIG. 2 illustrates a subassembly of a robotic actuator according to an embodiment of the present invention for extension actuation;

FIG. 3 illustrates a cross-sectional view of the subassembly shown in FIG. 2;

FIG. 4 illustrates a subassembly of a robotic actuator according to another embodiment for bending and extension actuation;

FIG. 5 illustrates a cross-sectional view of the subassembly shown in FIG. 4;

FIG. 6 illustrates a robotic actuator according to another embodiment of the present invention;

FIG. 7 illustrates a robotic actuator according to yet another embodiment;

FIG. 8 is a graph illustrating relative extension ratios of robotic actuators constituted by corrugated sleeves of TPU Shore hardness 60A, 85A and 95A;

FIGS. 9-10 are graphs illustrating bending profiles of the robotic actuators constituted by corrugated sleeves of TPU Shore hardness 85A and 95A, with pressure being incremented at a rate of 5 kPa;

FIG. 11 is a graph illustrating pressure-curvature relationship of bending of the robotic actuators constituted by corrugated sleeves of TPU Shore hardness 60A, 85A and 95A;

FIG. 12 is a graph illustrating blocked force output by bending the robotic actuators constituted by corrugated sleeves of TPU Shore hardness 60A, 85A and 95A with cylindrical bladders;

FIG. 13 is graph illustrating comparative blocked force output by bending the robotic actuators constituted by corrugated sleeves of TPU Shore hardness 60A and 85A with cylindrical and hemispherical bladders;

FIG. 14 is a graph illustrating blocked force output by extending the robotic actuators constituted by corrugated sleeves of TPU Shore hardness 85A and 95A; and

FIGS. 15 and 16 are pictures illustrating a use of the robotic actuator as an elbow assistive mechanism.

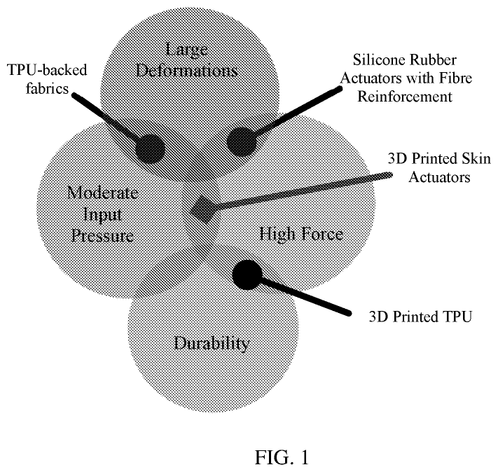

### DETAILED DESCRIPTION

One or more specific and alternative embodiments of the present invention will now be described with reference to the attached drawings. It shall be apparent to one skilled in the art, however, that this invention may be practised without such specific details. Some of the details may not be described at length so as not to obscure the invention. For ease of reference, common reference numerals or series of numerals will be used throughout the figures when referring to the same or similar features common to the figures.

The terms “skin” and “corrugated sleeve” are used to refer to a component disposed external to a bladder to constrain deformation, movement, and/or operation of the soft robotic actuator of the present invention;

The term “bladder” is used to refer to an internal component of the soft robotic actuator that receives a fluid medium to controllably deform the skin or corrugated sleeve surrounding the bladder;

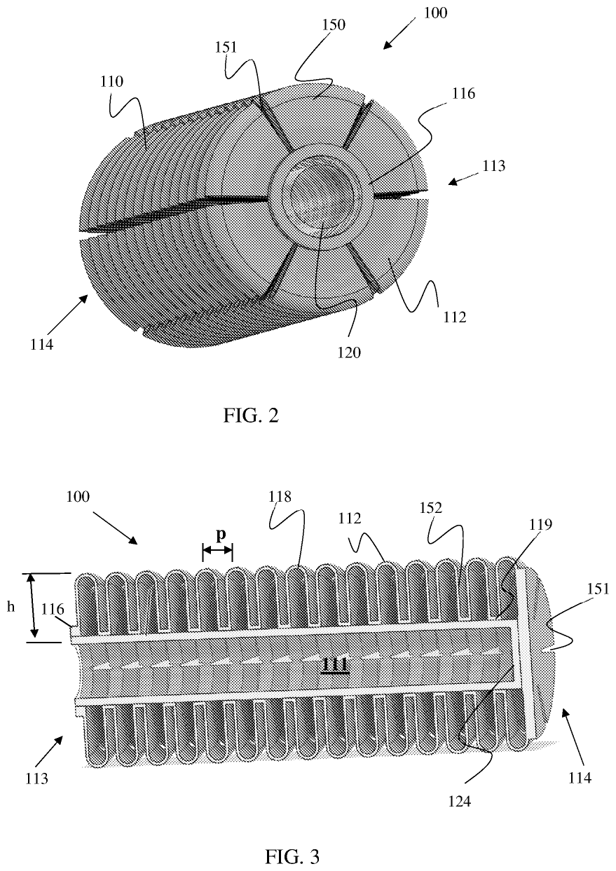

FIGS. 2 to 7 show various embodiments of soft robotic actuators 300,400 and subassemblies 100,200 of components that make up the soft robotic actuators. In general, each of the robotic actuators 300,400 or subassemblies 100,200 include two modules, namely, a corrugated sleeve 110,210 and a bladder 120,220. The bladder 120,220 is made of a low stiffness material (such as, silicone rubber) and is hermetically sealed, whilst the corrugated sleeve 110,210 is made of a higher stiffness material (such as, thermoplastic urethane (TPU)) than the bladder material and serves as an external skin to constrain deformations (extension and/or bending) of the bladder 120,220 when a fluid medium is supplied into the bladder. With the corrugated sleeve 110,210 and the bladder 120,220 being modular components, the robotic actuators thus obtained become reconfigurable according to a desired motion and a desired output force by inflating/deflating the bladder and deforming the corrugated sleeve.

As shown in FIGS. 2 and 3, the corrugated sleeve 110 is formed by a plurality of circular folds 112, like that of an accordion, and form a corrugated, cylindrical body with a substantially interior passageway 111. The passageway 111 extends from a first end 113 to a second end 114 of the corrugated sleeve 110, with the folds 112 of the corrugated sleeve 110 being fabricated with dimensions and being arranged in a geometrical configuration. The bladder 120 is detachably inserted inside the passageway 111 and, preferably, extends from the first end 113 to the second end 114 inside the corrugated sleeve 110. In a use application, a fluid medium is supplied into the bladder 120 under pressure through a tubing opening located at one end of the hermetically sealed bladder 120 to reversibly inflate the bladder 120; inflation by the fluid medium causes the bladder 120 to press against and to deform the corrugated sleeve 110, thereby generating a force output and extension of the robotic actuator, which is constituted by the corrugated sleeve 110 and the bladder 120. The fluid medium can be pneumatic- or hydraulic-based. The pneumatic medium may be atmospheric air, nitrogen, carbon dioxide, argon, helium, or any gas, employed to inflate the bladder 120. The hydraulic medium may be water, glycol, oil and so on.

As shown in FIGS. 2 and 3, the outmost edges 118 of the folds 112 are curved or arched in shape and collectively define the outer surface of the corrugated, cylindrical sleeve 110; likewise, the innermost edges 119 of the folds 112 are curved or arched and collectively define the passageway 111. In one embodiment, each fold 112 has a wall 152 thickness of substantially 0.5 to 2 mm, which is relatively lower than that of known actuators; for example, the corrugated folds 112 are 3D printed and the wall 152 thickness is substantially 2× the size of the 3D printing nozzle. In another embodiment, the inner most edges 119 of the folds 112 are flat and thus provide a larger area of contact with the bladder 120.

In one embodiment, the folds 112 are formed at a regular pitch p; the pitch p may be substantially 8 mm, but this is not so restricted; the pitch p may vary with the wall 152 thickness and may depend on characteristics of a 3D printer that is employed to fabricate the corrugated sleeve 110. When the pitch p is regular, the widths of the folds 112 along the outer surface and along the passageway 111 may be substantially the same; in another embodiment, even when the pitch p is regular, the widths of the folds 112 along the outer surface and along the passageway 111 may differ; preferably, the width of the folds 112 along the outer surface of the corrugated sleeve 110 is larger than the fold width along the passageway 111. An annular height h of the folds, as seen in FIG. 3, may be about 12 mm, or range from about 10 to 15 mm, depending on the application of the robotic actuator 300,400.

In FIG. 3, the first end 113 of the corrugated sleeve 110 is shown to be open, whilst the second end 114 is closed. An edge 116 around the open end 113 of the corrugated sleeve 110 is raised, for example, to reinforce the open end of the corrugated sleeve 110.

The bladder 120 shown in FIGS. 2 and 3 is elongate and is dimensioned to fit snugly inside the passageway 111 of the corrugated sleeve 110. The bladder wall thickness may range from substantially 2 to 4 mm.

The bladder 120 is made of a low stiffness material (such as, silicone rubber) and is hermitically sealed; the corrugated sleeve 110 is made of a higher stiffness, to provide an outer skin to constrain inflation of the bladder 120; in this manner, the corrugated sleeve 110 of the robotic actuators 300,400 thus configured is free from the need of being hermitically sealed, hence permitting the corrugated sleeve 110 to be fabricated at a reduced thickness, compared to known robotic actuators formed with a single thermoplastic polyurethane (TPU). The reduced thickness subsequently renders the assembled robotic actuator 300,400 to be operable under lower fluid pressures as the corrugated sleeve of lower wall 152 thickness requires less energy for deformation.

In another embodiment, the folds 112 are formed with longitudinal fissures 151 to form another geometric configuration of a corrugated sleeve 110a for constituting a robotic actuator. In one embodiment, the fissures 151 radiate radially from the passageway 111, are spaced regular and divide the corrugated sleeve 110a into regular sectors 150; the fissures 151 reduce the stiffness of extension of the corrugated sleeve 110a; with this geometric configuration, when the bladder 120 is inflated, the corrugated sleeve 110a deforms by extending in a substantially longitudinally direction at a lower fluid pressure than compared to a corrugated sleeve 110 without the fissures. In another embodiment, the fissures 151 are formed in a non-regular array about the passageway 111 and the corrugated sleeve 110b thus obtained is configured to both extend and bend, where bending is towards the side where the fissures 151 are located (but this is not illustrated in a figure).

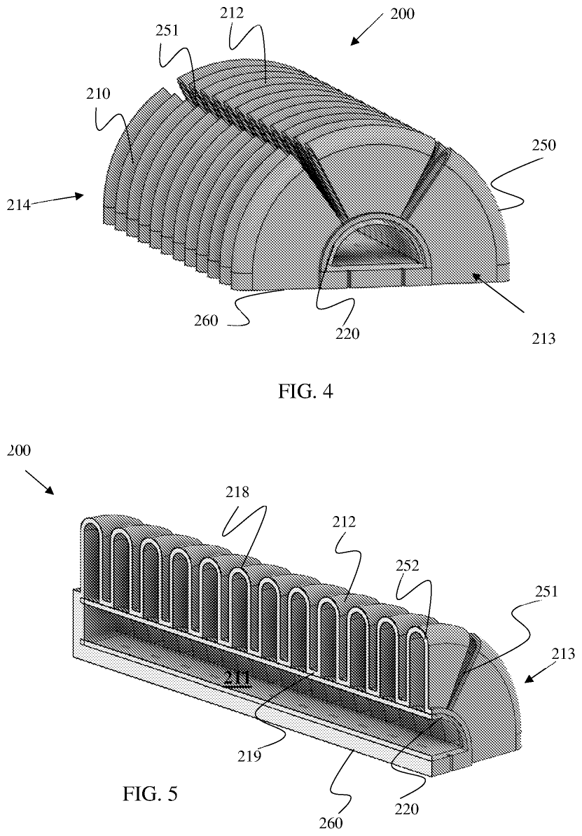

FIG. 4 shows a subassembly 200 obtained by combining a corrugated sleeve 210 and a bladder 220, whilst FIG. 5 shows a cut-section to show the interior arrangements. A robotic actuator using the subassembly 200 is configured to produce a greater degree of bending compared with the robotic actuator employing the above corrugated sleeve 110,110a,110b. As shown in FIG. 4, the corrugated sleeve 210 is a substantially semi-cylindrical in shape and forms a semi-cylindrical corrugated body 210. A flat planar surface 260 runs lengthwise along the corrugated sleeve 210 rendering an interior passageway 211 inside the corrugated sleeve also semi-cylindrical in shape. As the corrugated sleeve 210 with folds 212 forming the corrugations can flex more in relation to the flat surface 260, deformation of the corrugated sleeve 210 leads to bending towards flat surface 260 upon inflation of a bladder 220 disposed in the interior passageway 211.

In another embodiment of the corrugated sleeve 210a, longitudinal fissures 251 are formed, like the fissures described above, to reduce the extension stiffness of the corrugated sleeve 210a or to increase the flexibility of the resulting subassembly 200a. The nature of fabricating the folds and the fissures are as described above, and no further description is provided.

In a variation, the flat surface 260 of the corrugated sleeve 210,210a is slightly curved. In a further variation, the flat surface 260 may be fabricated with varied thickness or stiffness at different portions for regulating the ratio and degree of bending motion. According to the above embodiment and variations, a cylindrically shaped bladder 120 as described above can be fitted inside the passageway 211. Preferably, a bladder 220 with a semi-cylindrical shape is used: the bladder 220 that is semi-cylindrical shaped with a flat facade is able to fit snugly into the passageway 211 and thus provides more effective control on the extension and/or bending of the corrugated sleeve 210,210a.

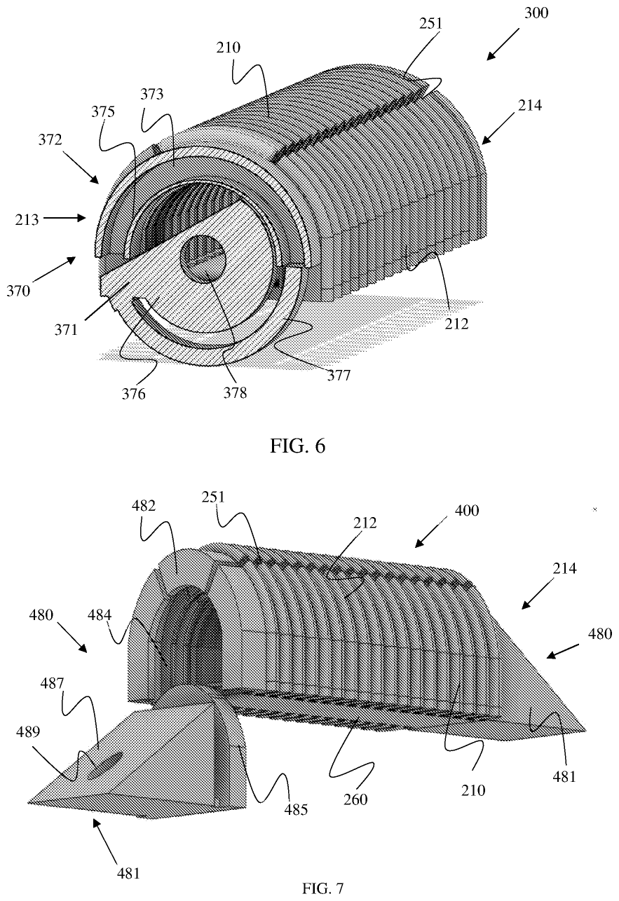

FIG. 6 shows a robotic actuator 300 according to an embodiment of the present invention. The robotic actuator 300 is made up of the above subassembly 200,200a and a lock member 370 located at a first end 213, with a second end 214 of the corrugated sleeve 210,210a being closed (similarly shown in FIG. 3). In a use configuration, two or more corrugated sleeves 210,210a may be connected in series and a distal end may be equipped with an end effector.

The lock member 370 is provided to removably secure the bladder 220 inside the corrugated sleeve 210,210a. As shown, the lock member 370 is made up of a male connector 371 in rotatory engagement with a female connector 372. Preferably, the female connector 372 is a substantially semi-annular member located on an end wall of the corrugated sleeve 210,210a and is formed with an annular trench 373. The male connector 371 is a semi-circular member 376, connected to an end of the bladder 220, and has a part-circular arm 377 that engages with the annular trench 373. The part-circular arm 377 has a fixed end extending from the semi-circular member 376 and the arm 377 hovers along part of the circular edge of the semi-circular member 376; as a result, a rift is formed between the semi-circular member 376 and the part-circular arm 377: a width of the rift is larger than a thickness of an inner sidewall 375 around the annular trench 373 so that the part-circular arm 377 can be rotatably engaged inside the annular trench 373 to facilitate the removeable securing of the bladder 220 inside the corrugated sleeve 210,210a. In one embodiment, both the male connector 371 and the female connector 372 are formed from the thermoplastic polyurethane (TPU) material used to form the corrugated sleeve 210,210a. It is also possible the male connector and the female connector are formed with a TPU with a higher Shore hardness value.

As shown in FIG. 6, the male connector 371 is provided with a through opening 378, which receives a tubing for supplying a pressurised fluid medium into the bladder 220. In a variation, the opening 378 is arranged to coincide with an opening of the bladder 220 for supplying the fluid medium during operation of the robotic actuator 300. It is also possible that the opening 378 is provided to receive both a tubing conveying the fluid medium and wires from sensors that may be used to monitor extension and bending of the robotic actuator 300 or any accompanying end effector.

FIG. 7 shows a robotic actuator 400 according to another embodiment of the present invention. The robotic actuator 400 is made up of the above subassembly 200,200a and a lock member 480 disposed at each of two ends of the subassembly. In another embodiment, a robotic actuator may be configured with two or more subassemblies connected in series and the lock member 480 is connected to one end, whilst the opposite end may be equipped with an end effector.

Each lock member 480 is made up of a male fastener 481 being connected to the bladder 220 and a female fastener 482 being connected to an end the corrugated sleeve 210,210a. The female fastener 482 is U-shaped and is formed with a tough 484. The tough 484 is accessible via a side opening for slidingly receiving and engaging a matching plate member 485 of the male connector 481. Whilst not shown, the plate member 485 is connected to the bladder 220, so that the male connector 481 slidingly engageable with the female connector 482 to form an assembly of the robotic actuator 400.

In FIG. 7, the male connector 481 is shown substantially as a triangular block 487 with the plate member 485 being located at a face edge. On another face edge of the male connector 481, a through hole 489 is provided for communicating a fluid medium into the bladder 220 or for running sensor wires into the passageway 211. For example, in an application, such as an elbow assistive mechanism for patient rehabilitation, the male connectors 481 are used as mounting points to a patient's limbs, a prothesis or an exoskeleton.

### Performance Tests of the Above Robotic Actuators:

Conventional robotic actuators using a single TPU material require high input pressure to generate deformation due to the high elastic modulus of TPU. Although this enables the generation of higher output forces, a large amount of energy is taken up in the deformation of the known robotic actuator itself. In the present invention, this limitation is circumvented by using the corrugated sleeve 110,110a,110b,210,210a based on TPU with the internal bladder 120,220 serving as a hermetic seal for containing the fluid medium. Thus, the bladder 120,220 is configured for transferring the force onto the corrugated sleeve 110,110a, 110b,210,210a, which serves as a programmable or controllable constraint. The corrugated sleeve 110,110a,110b,210,210a can thus be manufactured in much more complex configurations and can be separated due to the modular configuration. This allows a user to change the bladder 120,220 and the corrugated sleeve 110,110a,110b,210,210a combinations to alter the properties of the robotic actuators 300,400. The present invention illustrates bending and extension as two actuation modes of the robotic actuators. These two modes are the most used actuation modes for soft robotics applications, which may range from robotic locomotion to healthcare and rehabilitation. As described above, each robotic actuator 300,400 is made up of combinations of at least three modular components. The first component is the corrugated sleeve body 110,110a,110b,210,210a manufactured using 3D-printed thermoplastic polyurethane (TPU). The corrugated sleeve body controls the deformation profile of the robotic actuator 300,400. The second component, the bladder 120,220, is moulded from silicone rubber (obtainable from DragonSkin-10, Smooth-On, USA). The bladder 120,220 serves as a hermetic seal. The third component is a 3D printed TPU lock member 370,480 which enables the bladder 120,220 to be removeably secured inside the corrugated sleeve body 110,110a, 110b,210,210a; the lock member 370,480 also serves to route the fluid medium into the bladder; in addition, the lock member 480 also serves as a mounting point for the robotic actuator 400. The TPU material filament is BCN3D TPU 95A (since discontinued), which can be obtained from Ninajflex NinjaTek 85A or Polyflex Polymaker 95A, or X60, which can be obtained from Diabase Engineering, 60A.

Blue markers were placed on the corrugated sleeves of the robotic actuators 300,400 to capture deformation data on a tracking software. The robotic actuators were then mounted vertically on a retort stand. The fluid pressure was increased in 5 kPa increments. A tracker (Open-Source Physics) was used to process the video and extract coordinates of the robotic actuator deformations; the raw data thus obtained were then processed using Matlab to plot deformations (extension and bending) of the robotic actuator.

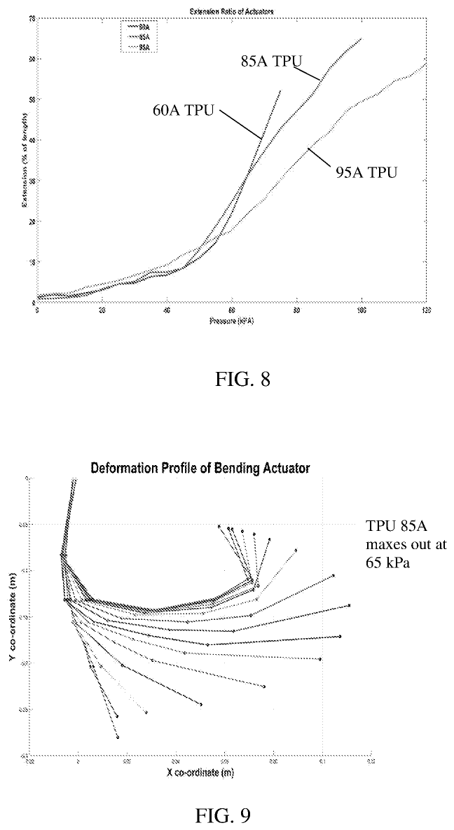

FIG. 8 shows extension ratios of the corrugated sleeve 110,110a constituting the robotic actuators, with the corrugated sleeve being fabricated with TPU of Shore hardness 60A, 85A and 95A. Initially, the extension ratio changes of the three TPU materials are similar. This is likely due to the bladder 120 initially expanding to contact the corrugated sleeve at the passageway 111 when the pressure is about 45 kPa or less. From about 60 kPa pressure, the corrugated sleeve made of softer TPU materials start to deform more. Since the Young's Moduli of the TPU material increase in line with their Shore hardness, it is evident that extension of the 60A corrugated sleeve is the largest for a given pressure, while that of the 95A corrugated sleeve is the smallest.

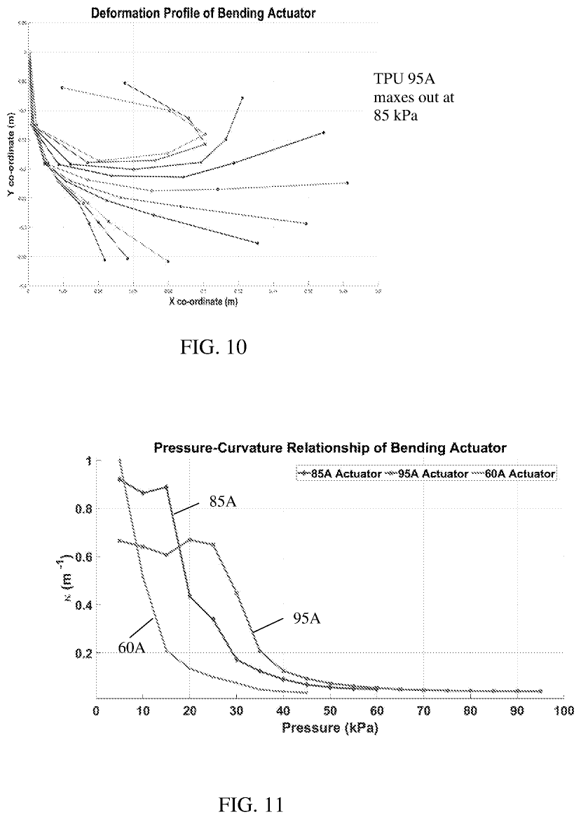

The above robotic actuators 300,400 with the corrugated sleeve 210,210a being also fabricated with TPU Shore harness 60A, 85A and 95A are tested for bending performance. The fluid pressure was incremented at 5 kPa interval until the free end of the robotic actuator curled up and touched the end that is held at the retort stand. FIG. 9 shows the corrugated sleeve made of 85A TPU and the bending maxes out at about 65 kPa. In contrast, FIG. 10 shows the robotic actuator made with corrugated sleeve of TPU 95A curled up and the bending maxes out at about 85 kPa, which is evident for the higher stiffness of the 95A TPU. Another plot of pressure-bending curvature characteristics of the 85A and 95A TPU are shown in FIG. 11; ignoring the initial inaccuracy at about 20 kPa and below, the robotic actuator made with 95A TPU exhibits higher stiffness and the 85A TPU maxes out at a lower pressure of about 50 kPa. The above test results show that the actuation pressure of the above robotic actuators is less than about 150 kPa, which is a desirable pressure for a wearable actuator system.

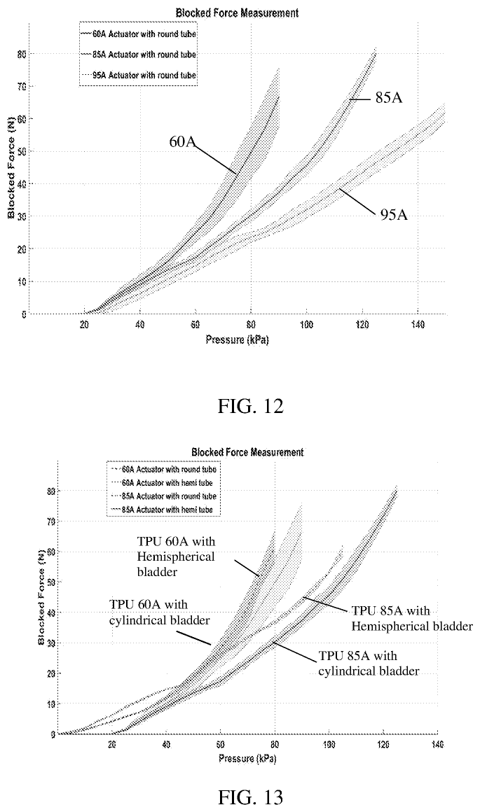

Force output of a robotic actuators also determines their applications; for example, a wearable assistive device represents its ability of the robotic actuator to perform a task. A Vertical Automatic Handy Tester (JSV H1000, Measuring Instrument Technology, Singapore) with a force gauge was used to measure the force output of the above robotic actuators. The robotic actuators made with corrugated sleeve 110,110a,110b of 60A, 85A and 95A TPU and equipped with cylindrical bladders 120 were mounted in an inverted condition to apply a compressive force on the force gauge against the force of gravity. The actuator is then pressurized in increments of 5 kPa; three data sets were taken for each robotic actuator. The mean and variance of the force output were plotted. FIG. 12 shows the blocked force output by actuating the robotic actuators in the bending mode. From FIG. 12, it is observed that the lower stiffness actuator provided higher force output at the same pressure as compared to the higher stiffness ones. This is because less energy was used to bend the robotic actuator having a lower stiffness, thereby, allowing the robotic actuator of lower stiffness to transfer a higher proportion of the fluid pressure to useful work. The higher stiffness actuators could tolerate higher pressures owing to their correspondingly higher Young's Moduli. Despite lacking the force output for the same pressures as their softer counterparts, the robotic actuator exhibiting higher stiffness of TPU 85A and 95A are routinely chosen for applications since they generate higher peak forces with less mechanical failure.

FIG. 13 shows the blocked force profiles of bending robotic actuators 300,400 using the cylindrical bladder 120 and semi-cylindrical bladders (Hemi-tube) 220. In particular, 60A and 85A TPU corrugated sleeves 210,210a are used and the blocked force curves are plotted. From the test results, the robotic actuators using the semi-cylindrical bladder 220 of the same material generate about 20% higher force at the same pressure. This is because the cylindrical bladder 120 must first expand to contact the semi-cylindrical passageway 211, and thus the initial force transfer does not perform useful work. Thus, a semi-cylindrical bladder 220 to fit snugly inside the corrugated sleeve 210,210a is a better choice to use. This snug fit of the bladder also translated to lower variance between the tests using the semi-cylindrical bladder 220.

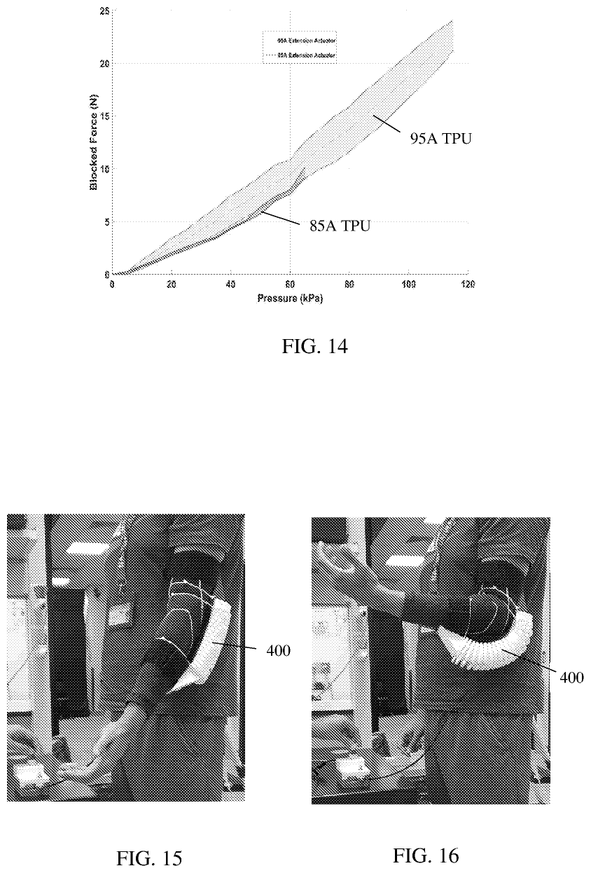

FIG. 14 shows the blocked force profiles of extension robotic actuators 300,400 using the cylindrical bladder 120 and the cylindrical sleeve 110 made with 85A and 95A TPU. From FIG. 14, it is seen that 95A TPU higher stiffness actuator transfer larger forces at pressures higher than 10 kPa. This is likely because the extension actuator with the cylindrical bladder 120 has less transmission loss between the bladder and the cylindrical sleeve 110, and 10 kPa is the initial pressure for the cylindrical bladder to expand and make close contact along the cylindrical passageway 111 inside the cylindrical sleeve 110.

FIG. 7 shows the robotic actuator 400 which can be used to assist an elbow exoskeleton. FIGS. 15 and 16 illustrate an application of the robotic actuator 400 at an elbow where the lock members 480 at both the ends are used as mounting points. The ability of the robotic actuator 400 to generate sufficient high force is desirable in such an assistive mechanism. The elbow is an upper body joint that requires high assistive torque, usually as it is load bearing and experiences a large moment arm from the hand, where an object is grasped.

While specific embodiments have been described and illustrated, it is understood that many changes, modifications, variations and combinations of variations disclosed in the text description and drawings thereof could be made to the present invention without departing from the scope of the present invention. For example, a fabric strip can be attached to a side of the bladder 120,220 to control bending of the bladder; it is also possible that the fabric strip be attached to the flat portion 260 of the semi-cylindrical sleeve to control bending of the semi-cylindrical sleeve 210,210a. The fabric strip is flexible but has a tensile stiffness higher than the TPU.

1.-24. (canceled)

25. A robotic actuator comprising:
a corrugated sleeve formed with corrugating folds extending from a first end to a second end and defining an interior passageway; and
a bladder being detachably disposed inside the interior passageway of the corrugated sleeve by a lock member;
wherein, when a fluid medium is supplied into the bladder, the bladder is inflated, thereby compressing against the interior passageway, deforming the corrugated sleeve and providing a force output at the robotic actuator, and the bladder is reversible deflated when the fluid medium is released.

26. The robotic actuator according to claim 25, wherein the lock member comprises a female connector and a male connector being connected to the first or the second end of the corrugated sleeve, so that the female and male connectors are engageable or dis-engageable by a rotatory motion.

27. The robotic actuator according to claim 25, wherein the lock member comprises a female connector and a male connector being connected to the first or the second end of the corrugated sleeve, so that the female and male connectors are engageable or dis-engageable by a sliding motion.

28. The robotic actuator according to claim 25, wherein the corrugated sleeve is cylindrically shaped.

29. The robotic actuator according to claim 25, wherein the corrugated sleeve is substantially hemi-cylindrical shaped and comprises a flat surface, resulting in the interior passageway being substantially hemi-cylindrical.

30. The robotic actuator according to claim 25, wherein the bladder is shaped to fit substantially snugly into the interior passageway.

31. The robotic actuator according to claim 25, wherein the corrugating folds are spaced apart at a regular pitch distance.

32. The robotic actuator according to claim 25, wherein a wall of each corrugating fold at both an outside surface and the interior passageway is curved or arched shaped.

33. The robotic actuator according to claim 25, wherein a wall of each of the corrugating fold at the interior passageway is substantially flat.

34. The robotic actuator according to claim 32, wherein a width of each corrugating fold at the outside surface has a bigger dimension than a width of the corrugating fold at the interior passageway.

35. The robotic actuator according to claim 25, wherein radial fissures are formed radiating from the interior passageway and extending to both ends of the corrugated sleeve.

36. The robotic actuator according to claim 25, wherein the bladder is made of a material of lower stiffness than a material of the corrugated sleeve, so that the bladder is hermetically sealed and is inflatable by the fluid medium, whilst the corrugated sleeve controls extension or bending of the robotic actuator caused by inflating the bladder.

37. The robotic actuator according to claim 36, wherein the bladder material is silicone rubber, whilst the material of the corrugated sleeve is thermoplastic urethane (TPU).

38. The robotic actuator according to claim 25, further comprising a fabric strip that is attachable along a side of the bladder to control a direction of bending of the bladder.

39. The robotic actuator according to claim 29, further comprising a fabric strip that is attachable onto the flat surface of the hemi-cylindrical sleeve.

40. The robotic actuator according to claim 29, wherein a material along the flat surface is formed with a varied stiffness, so as to control bending of the hemi-cylindrical sleeve.

41. A method for configuring extension or bending of a robotic actuator, comprising the steps of:
configuring a corrugated sleeve by forming corrugating folds to extend from a first end to a second end, and defining an interior passageway; and
removeably securing a bladder inside the corrugated sleeve by connecting a lock member to the first or the second end of the bladder and a corresponding end of the corrugated sleeve, wherein the bladder is hermitically sealed to receive a fluid medium, wherein the bladder is formed from a material of lower stiffness compared to a material of the corrugated sleeve;
wherein, when the fluid medium is supplied into the bladder, inflation of the bladder acts on the interior passageway, thereby causing a force output and extension of the robotic actuator.

42. The method according to claim 41, further comprises forming a fissure along the corrugated sleeve to alter the stiffness of the corrugated sleeve, so that the force output is increased and the robotic actuator bends away from location of the fissure.

43. The method according to claim 41, further comprises forming the corrugated sleeve with a flat surface to obtain a hemi-cylindrical passageway, so as to control the bending stiffness of the corrugated sleeve towards the flat surface.

44. The method according to claim 43, wherein controlling the bending stiffness about the flat surface of the corrugated sleeve is implemented by varying a material stiffness along the flat surface or attaching a fabric strip along the flat surface.

45. A method for configuring an assistive mechanism using the robotic actuator according to claim 40, comprising the steps of:
inserting a bladder inside a corrugated sleeve, wherein the bladder is hermitically sealed to receive a fluid medium and forming the bladder from a material of lower stiffness compared to a material of the corrugated sleeve;
removeably securing one end of the bladder to an associated end of the corrugated sleeve with a lock member;
mounting an opposite end of the corrugated sleeve with another lock member; and
securing each of the lock members to a limb constituting a joint;
wherein inflating the bladder creates a force output, and resulting extension and bending of the robotic actuator creates an assistive motion about the joint, whilst deflating the bladder reverses motions of the robotic actuator.

46. The method according to claim 45, wherein the joint is constituted with a prothesis or an exoskeleton.
